(19) United States
(12) Patent Application Publication
(10) Pub. No.: US 2026/0202414 A1
(43) Pub. Date: Jul. 16, 2026

(54) PROTEIN COMPLEXES FOR PROXIMITY-BASED LABELING OF INTRACELLULAR MICROENVIRONMENTS

(71) Applicant: THE TRUSTEES OF PRINCETON UNIVERSITY, Princeton, NJ (US)

(72) Inventors: David W.C. MacMillan, Princeton, NJ (US); Benito Buksh, Princeton, NJ (US); Holt Sakai, Princeton, NJ (US); Chun Li, Princeton, NJ (US); Steven Douglas Knutson, Princeton, NJ (US); Johnny Wang, Princeton, NJ (US)

(21) Appl. No.: 19/134,520

(22) PCT Filed: Nov. 30, 2023
(86) PCT No.: PCT/US2023/081832

§ 371 Date: May 30, 2025

Related U.S. Application Data
(60) Provisional application No. 63/428,899, filed on Nov. 30, 2022.

(51) Int. Cl. G01N 33/58

(52) U.S. Cl. CPC G01N 33/581; G01N 2333/9108; G01N 2333/914

(57) ABSTRACT

Protein complexes and associated methods are described herein for proximity-based labeling of various intracellular or intercellular microenvironments. In some embodiments, a protein complex comprises a protein substrate having an ubiquitin E3 ligase associated therewith, wherein a transition metal complex is coupled to the protein substrate. In some embodiments, one or more intermediate molecular species, such as a small molecule and/or protein, reside between the ubiquitin E3 ligase and protein substrate.

### RELATED APPLICATION DATA

The present application claims priority pursuant to Article 8 of the Patent Cooperation treaty to U.S. Provisional Patent Application Ser. No. 63/428,899 filed Nov. 30, 2022 which is incorporated herein by reference in its entirety.

### FIELD

The present invention relates to compositions and methods for intracellular proximity-based labeling and, in particular, to protein complexes for proximity-based labeling of various intracellular microenvironments.

### BACKGROUND

Protein proximity labeling has emerged as a powerful approach for profiling protein interaction networks. The ability to label associated or bystander proteins through proximity labeling can have important implications on further understanding the cellular environment and biological role of a protein of interest. Current proximity labeling methods all involve the use of enzyme-generated reactive intermediates that label neighboring proteins on a few select amino acid residues through diffusion or physical contact. Despite the transformative impact of this technology, the inherent stability of these reactive intermediates such as phenoxy radicals (t_{1/2}>100 μs) through peroxidase activation or biotin-AMP (t_{1/2}>60 s) through biotin ligases can promote diffusion far from their point of origin. As a result, these enzyme-generated reactive intermediates pose a challenge to profiling within tight micro-environments. Furthermore, the large enzyme size, the dependency on certain amino acids for labeling, and the inability to temporally control these labeling systems present additional challenges for profiling within confined spatial regions. Given these limitations, new approaches for proximity-based labeling are needed.

### SUMMARY

In view of the foregoing disadvantages, protein complexes and associated methods are described herein for proximity-based labeling of various intracellular or intercellular microenvironments. In some embodiments, a protein complex comprises a protein substrate having an ubiquitin E3 ligase associated therewith, wherein a transition metal complex is coupled to the protein substrate. In some embodiments, one or more intermediate molecular species, such as a small molecule and/or protein, reside between the ubiquitin E3 ligase and protein substrate.

Moreover, the transition metal complex can be coupled to the protein substrate via a variety of mechanisms or architectures. In some embodiments, the transition metal complex is bonded to a haloalkane dehalogenase, the haloalkane dehalogenase coupled to the protein substrate. Alternatively, the transition metal complex is coupled to the protein substrate via protein-trans splicing, the transition metal complex being initially coupled to a split intein. The split intein carrying the transition metal complex can be an N-intein or an C-intein. In further embodiments, the transition metal complex is bonded to a derivatized or unnatural amino acid of the protein substrate. Suitable click chemistry moieties of the transition metal complex and/or derivatized/unnatural amino acid, for example, can be selected from the group consisting of DBCO, BCN, TCO, tetrazine, alkyne and azide. In some embodiments, the transition metal complex is of Formula (I):

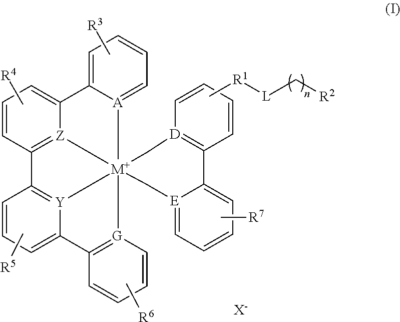

wherein Mis a transition metal;
wherein A, D, E, G, Y and Z are independently selected from C and N;
wherein R³-R⁷ each represent one to four optional ring substituents, each of the one to four optional ring substituents independently selected from the group consisting of alkyl, heteroalkyl, haloalkyl, haloalkenyl, halo, hydroxy, alkoxy, amine, amide, ether, —C(O)O⁻, —C(O)OR⁸, and —R⁹OH, wherein R⁸ is selected from the group consisting of hydrogen and alkyl, and R⁹ is alkyl;
wherein R¹ is selected from the group consisting of a direct bond, alkylene, alkenylene, cycloaklylene, cycloalkenylene, arylene, heteroalkylene, heteroalkenylene, heterocyclene, and heteroarylene;
wherein L is an optional linking moiety selected from the group consisting of amide, ester, sulfonamide, sulfonate, carbamate, and urea; and
R² is selected from the group consisting of alkyne, amine, protected amine, azide, hydrazide, aryl, heteroaryl, cycloalkyl, cycloalkenyl, cycloalkylnyl, heterocyclyl, hydroxy, carboxyl, halo, alkoxy, maleimide, —C(O)H, —C(O)OR⁸, —OS(O₂)R⁹, thiol, biotin, oxyamine, and haloalkyl, wherein R⁸ and R⁹ are independently selected from the group consisting of alkyl, haloalkyl, aryl, haloaryl, N-succinimidyl, and N-succinimidyl ester; and wherein X⁻ is a counterion, and n is an integer from 0 to 20.

In another aspect, methods of characterizing protein degradation or proteolysis are described herein. In some embodiments, a method of characterizing protein degradation comprises providing a protein substrate having a transition metal complex associated therewith, and activating a protein labeling agent to a reactive intermediate with the transition metal complex. The reactive intermediate couples to an ubiquitin E3 ligase forming a complex with the protein substrate. The transition metal complex can be associated with the protein substrate by any architecture described herein. The transition metal complex can have electronic structure for energy transfer to a protein labeling agent to produce the reactive intermediate. In some embodiments, the energy transfer is Dexter energy transfer or electron transfer. Energy transfer to the protein labeling agent can originate from an excited state of the transition metal complex electronic structure, in some embodiments. The excited state of the complex, for example, can be a singlet excited state or triplet excited state. Alternatively, energy transfer to the protein labeling agent, including electron transfer, may originate from a ground state of the transition metal complex electronic structure. The transition metal complex can be of Formula (I) provided above.

The diffusion radius of the reactive intermediate can be tailored to specific microenvironment mapping (proximity-based labeling) considerations, and can be limited to the nanometer scale. Accordingly, the reactive intermediate will react or crosslink with a protein or other biomolecule within the diffusion radius or be quenched by the surrounding environment if no protein or biomolecule is present. High resolution of the environment local to the protein substrate can be mapped via concerted effort between the transition metal complex and protein labeling agent. In this way, the ubiquitin E3 ligase forming a complex with the protein substrate can be labeled or determined. Other molecular species forming the complex can also be elucidated with the labeling agent, including small molecules and/or proteins recruiting or linking the ubiquitin E3 ligase to the protein substrate.

In some embodiments, the method further comprises coupling a small molecule to the protein substrate, the small molecule providing attachment of the ubiquitin E3 ligase to the protein substrate thereby resulting in the protein complex. The reactive intermediate, in some embodiments, may also couple to the small molecule. Structural information derived from the ubiquitin E3 ligase forming a complex with the protein substrate and/or small molecule can be employed to develop one more additional small molecules operable to recruit the ubiquitin E3 ligase to different protein substrates. A library of molecular glues can be constructed for various E3 ligases and/or other molecules.

In another aspect, protein complexes and systems are described herein for mapping intracellular protein microenvironments. In some embodiments, a protein complex comprises a transition metal complex bonded to a haloalkane dehalogenase, the haloalkane dehalogenase coupled to a protein. Alternatively, the transition metal complex is coupled to the protein via protein-trans splicing, the transition metal complex being initially coupled to a split intein. The split intein carrying the transition metal complex can be an N-intein or an C-intein. In additional embodiments, the transition metal complex is bonded to a derivatized or unnatural amino acid of the protein. Suitable click chemistry moieties of the transition metal complex and/or derivatized amino acid can be selected from the group consisting of DBCO, BCN, TCO, tetrazine, alkyne and azide. The transition metal complex can be of Formula (I) herein.

A protein labeling agent or biomolecular labeling agent can work in conjunction with the transition metal complex to generate reactive intermediates, as described above. The reactive intermediate can label proteins and/or other chemical and/or biomolecular species in the local microenvironment of the protein complex.

In another aspect, methods of profiling microenvironments local to an intracellular protein are described herein. In some embodiments, a method comprises forming a protein complex comprising a transition metal complex coupled to the intracellular protein, and activating a protein labeling agent to a reactive intermediate with the transition metal complex. The reactive intermediate is coupled to a protein or other biomolecule within a predetermined radius of the protein complex. The protein complex, transition metal complex, and protein labeling agent can have any composition and/or properties described herein.

These and other embodiments are further described in the following detailed description.

### BRIEF DESCRIPTION OF THE DRAWINGS

FIG. 1 illustrates transition metal complexes described herein according to some embodiments.

FIG. 2 illustrates binding of a transition metal complex described herein to a protein via haloalkane dehalogenase, according to some embodiments.

FIG. 3 illustrates a process for binding a transition metal complex to a protein substrate via an unnatural amino acid and subsequently labeling with a protein labeling agent according to some embodiments.

FIG. 4 illustrates protein substrate labeling results employing the substrate bound transition metal complex of FIG. 3.

FIG. 5 and FIG. 6 illustrate a validation of a split intein splicing strategy with Histone H3.1 and iridium transition metal complex described herein.

FIG. 7 conceptually illustrates mapping of the ERα interactome post estradiol and fulvestrant binding, according to some embodiments.

FIGS. 8-11 conceptually illustrate identification of species participating in ERα degradation pathways elucidated by proximity labeling methods described herein, according to some embodiments.

FIG. 12 illustrates employing information obtained from protein interactome mapping methods described herein for the development of E3 ligase activators and molecular glues, according to some embodiments.

FIG. 13 is a volcano plot detailing the species identified in ERα interactome mapping post binding of estradiol and fulvestrant, according to some embodiments.

### DETAILED DESCRIPTION

Embodiments described herein can be understood more readily by reference to the following detailed description and examples and their previous and following descriptions. Elements, apparatus and methods described herein, however, are not limited to the specific embodiments presented in the detailed description and examples. It should be recognized that these embodiments are merely illustrative of the principles of the present invention. Numerous modifications and adaptations will be readily apparent to those of skill in the art without departing from the spirit and scope of the invention.

### Definitions

The term “alkyl” as used herein, alone or in combination, refers to a straight or branched saturated hydrocarbon group optionally substituted with one or more substituents. For example, an alkyl can be C₁-C₃₀ or C₁-C₁₈.

The term “alkenyl” as used herein, alone or in combination, refers to a straight or branched chain hydrocarbon group having at least one carbon-carbon double bond and optionally substituted with one or more substituents.

The term “alkynyl” as used herein, alone or in combination, refers to a straight or branched chain hydrocarbon group having at least one carbon-carbon triple bond and optionally substituted with one or more substituents.

The term “aryl” as used herein, alone or in combination, refers to an aromatic monocyclic or multicyclic ring system optionally substituted with one or more ring substituents.

The term “heteroaryl” as used herein, alone or in combination, refers to an aromatic monocyclic or multicyclic ring system in which one or more of the ring atoms is an element other than carbon, such as nitrogen, boron, oxygen and/or sulfur.

The term “heterocycle” as used herein, alone or in combination, refers to a mono- or multicyclic ring system in which one or more atoms of the ring system is an element other than carbon, such as boron, nitrogen, oxygen, and/or sulfur or phosphorus and wherein the ring system is optionally substituted with one or more ring substituents. The heterocyclic ring system may include aromatic and/or non-aromatic rings, including rings with one or more points of unsaturation.

The term “cycloalkyl” as used herein, alone or in combination, refers to a non-aromatic, mono- or multicyclic ring system optionally substituted with one or more ring substituents.

The term “heterocycloalkyl” as used herein, alone or in combination, refers to a non-aromatic, mono- or multicyclic ring system in which one or more of the atoms in the ring system is an element other than carbon, such as boron, nitrogen, oxygen, sulfur or phosphorus, alone or in combination, and wherein the ring system is optionally substituted with one or more ring substituents.

The term “alkoxy” as used herein, alone or in combination, refers to the moiety RO—, where R is alkyl, alkenyl, or aryl defined above.

The term “halo” as used herein, alone or in combination, refers to elements of Group VIIA of the Periodic Table (halogens). Depending on chemical environment, halo can be in a neutral or anionic state.

Terms not specifically defined herein are given their normal meaning in the art.

### I. Protein Complexes

Protein complexes and associated methods are described herein for proximity-based labeling of various intracellular or intercellular microenvironments. In some embodiments, a protein complex comprises a protein substrate having an ubiquitin E3 ligase associated therewith, wherein a transition metal complex is coupled to the protein substrate. In some embodiments, one or more intermediate molecular species, such as a small molecule and/or protein, reside between the ubiquitin E3 ligase and protein substrate. The small molecule and/or protein can serve as a linker between the ubiquitin E3 ligase and protein substrate in the formation of a ternary complex. The small molecule and/or protein, for example, can exhibit recognition motifs binding or recruiting the ubiquitin E3 ligase to the protein substrate. In some embodiments, the small molecule and/or protein is attached to the ubiquitin E3 ligase prior to forming the ternary complex with the protein substrate. Alternatively, the small molecule and/or protein is attached to the protein substrate prior to forming the ternary complex with the ubiquitin E3 ligase. In an additional embodiment, the protein substrate and ubiquitin E3 ligase can initially form an interaction, and the small molecule and/or protein subsequently binds to form the ternary complex.

As described herein, the transition metal complex can activate a protein labeling agent to a reactive intermediate, wherein the reactive intermediate couples to the ubiquitin E3 ligase of the ternary complex. The protein labeling agent can be any species consistent with the technical principles described herein. The protein labeling agent, in some embodiments, is a diazirine or an azide. As described herein, the transition metal complex can form a plurality of reactive intermediates permitting labeling a number of biomolecular species within a radius of the ternary complex. Accordingly, the reactive intermediate, in some embodiments, may also couple to the small molecule and/or protein binding the ubiquitin E3 ligase and protein substrate. Therefore, the ubiquitin E3 ligase and/or small molecule/protein of the ternary complex may be labeled with the protein labeling agent. Having the transition metal complex coupled to the protein substrate can permit profiling of microenvironments local to the protein substrate, including the identification of biomolecular species involved in the degradation or proteolysis of the protein substrate. In this way, the proteolytic pathway can be further elucidated and structural motifs recognized for developing a library small molecule species enabling binding of ubiquitin E3 ligases to the protein substrate and other proteins of interest.

In alternative embodiments, a protein complex comprises a protein substrate having an ubiquitin E3 ligase associated therewith, wherein a transition metal complex is coupled to the ubiquitin E3 ligase. In such embodiments, the protein substrate can be labeled with a protein labeling agent via the reactive intermediate. Moreover, any small molecule and/or protein forming a ternary complex with the substrate and ubiquitin E3 ligase can also be labeled with the protein labeling agent. Having the transition metal complex coupled to the ubiquitin E3 ligase can permit profiling of microenvironments local to the E3 ligase. In this way proteins interacting with the E3 ligase for ubiquitination can be identified. Such identification can discover previously unknown interactions, thereby expanding the use of E3 ligases for broader protein degradation applications.

Protein complexes described herein, in some embodiments, are located in the intracellular environment. In some embodiments, for example, a protein complex is located in the nucleus of the cell. Alternatively, protein complexes can be located in the cytoplasm or membranes of the cell. The transition metal complex can interact with the protein substrate or E3 ligase by covalent bonding. In other embodiments, the transition metal complex may interact with a protein substrate or E3 ligase via electrostatic interactions and/or van der Waals interactions.

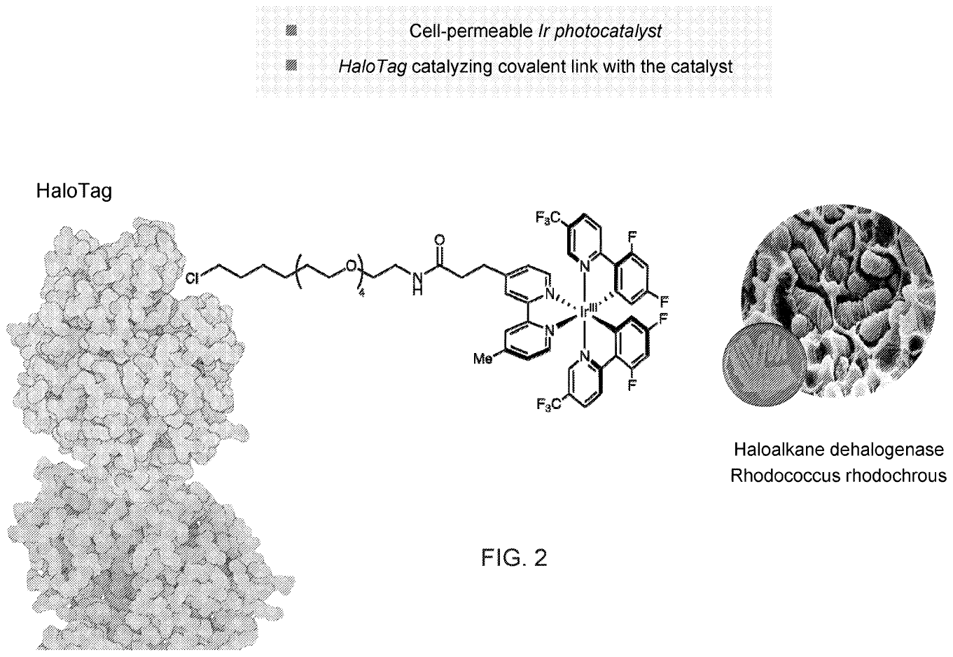

The transition metal complex, in some embodiments, is coupled to a protein substrate or E3 ligase of a protein complex via interaction with haloalkane dehalogenase (HaloTag). Haloalkane dehalogenase, for example, can be co-expressed with the protein substrate using standard cloning procedures. The transition metal complex can be functionalized with a haloalkane moiety for covalently binding with the haloalkane dehalogenase resulting in coupling of the protein of interest. FIG. 2 illustrates binding of a transition metal complex described herein to a protein via haloalkane dehalogenase, according to some embodiments.

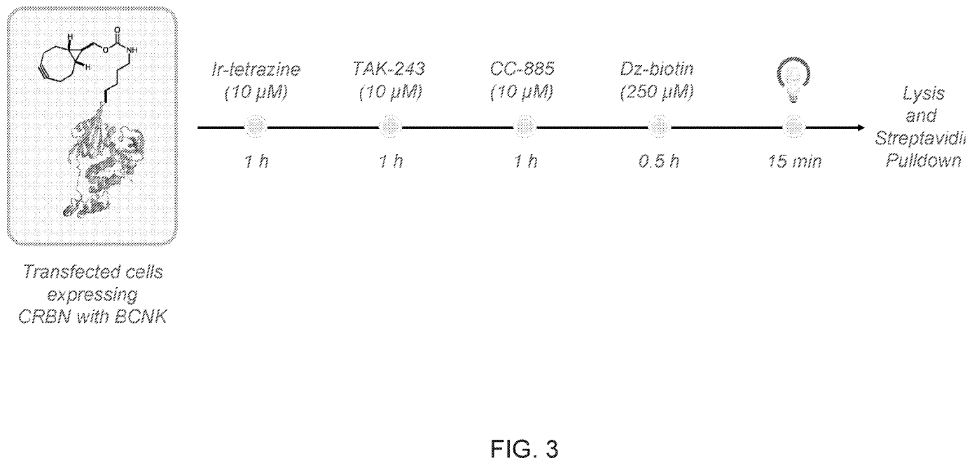

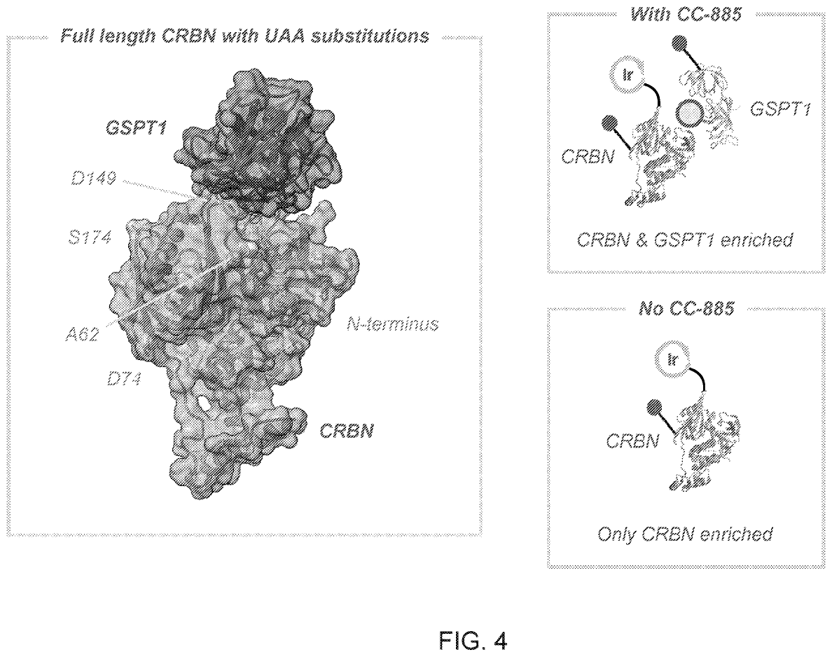

Alternatively, the transition metal complex can be bonded to a derivatized amino acid or unnatural amino acid of the protein substrate or E3 ligase. Suitable click chemistry moieties of the transition metal complex and/or unnatural amino acid can be selected from the group consisting of DBCO, BCN, TCO, tetrazine, alkyne and azide. FIG. 3 illustrates validation of such a binding strategy with cereblon (CRBN). Transfected cells were employed to express CRBN with BCNK. Iridium complex comprising a tetrazine moiety was introduced to bind the iridium complex to CRBN. CRBN modulator CC-885 was added to bind to CRBN, and diazo-biotin was added as a protein labeling agent. The iridium complex was irradiated to form a reactive intermediate through energy transfer to the diazo-biotin as described further herein. The reactive intermediate binding to biomolecular species within a predetermined radius of the CRBN-Ir. FIG. 4 illustrates the results of the labeling study. In the presence of CRBN and CC-885, CRBN and GSPT1 are enriched. In the absence of CC-885, only CRBN is enriched.

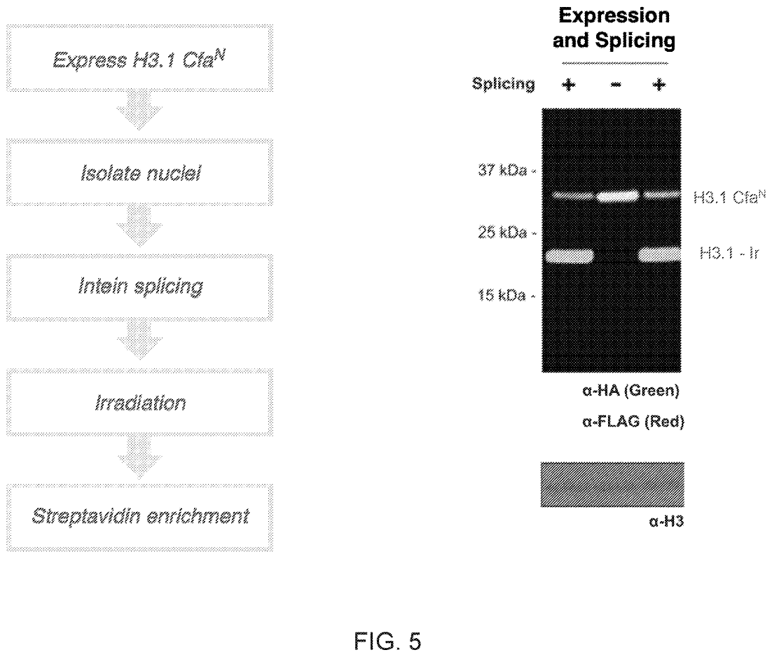

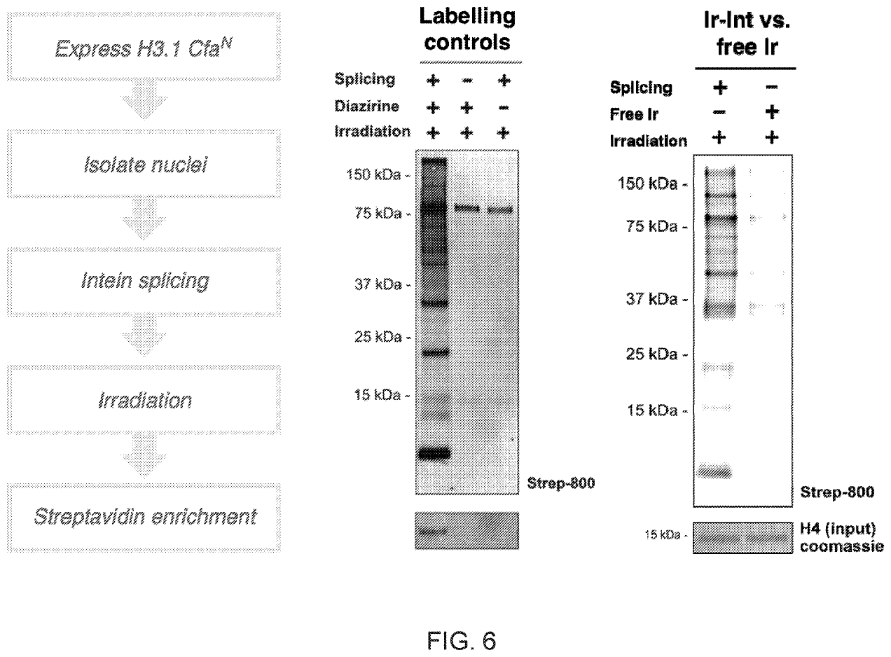

Moreover, in some embodiments, the transition metal complex is coupled to a protein substrate or E3 ligase via protein-trans splicing, the transition metal complex being initially coupled to a split intein. The split intein carrying the transition metal complex can be an N-intein or an C-intein. FIGS. 5 and 6 illustrate a validation of a split intein splicing strategy with Histone H3.1 and iridium transition metal complex described herein.

In some embodiments, the transition metal complex is of Formula I:

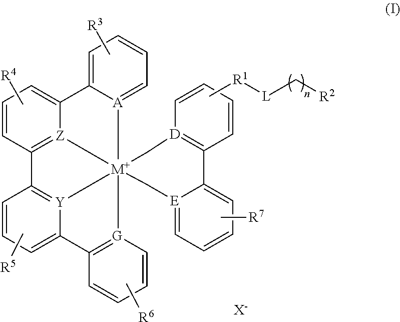

wherein M is a transition metal;
wherein A, D, E, G, Y and Z are independently selected from C and N;
wherein R³-R⁷ each represent one to four optional ring substituents, each of the one to four optional ring substituents independently selected from the group consisting of alkyl, heteroalkyl, haloalkyl, haloalkenyl, halo, hydroxy, alkoxy, amine, amide, ether, —C(O)O⁻, —C(O)OR⁸, and —R⁹OH, wherein R⁸ is selected from the group consisting of hydrogen and alkyl, and R⁹ is alkyl;
wherein R¹ is selected from the group consisting of a direct bond, alkylene, alkenylene, cycloaklylene, cycloalkenylene, arylene, heteroalkylene, heteroalkenylene, heterocyclene, and heteroarylene;
wherein L is an optional linking moiety selected from the group consisting of amide, ester, sulfonamide, sulfonate, carbamate, and urea; and
R² is selected from the group consisting of alkyne, amine, protected amine, azide, hydrazide, aryl, heteroaryl, cycloalkyl, cycloalkenyl, cycloalkylnyl, heterocyclyl, hydroxy, carboxyl, halo, alkoxy, maleimide, —C(O)H, —C(O)OR⁸, —OS(O₂)R⁹, thiol, biotin, oxyamine, and haloalkyl, wherein R⁸ and R⁹ are independently selected from the group consisting of alkyl, haloalkyl, aryl, haloaryl, N-succinimidyl, and N-succinimidyl ester; and wherein X is a counterion, and n is an integer from 0 to 20. As provided in Formula I, the linking moiety, L, is optional and, therefore, may not be present in some embodiments of the transition metal complex.

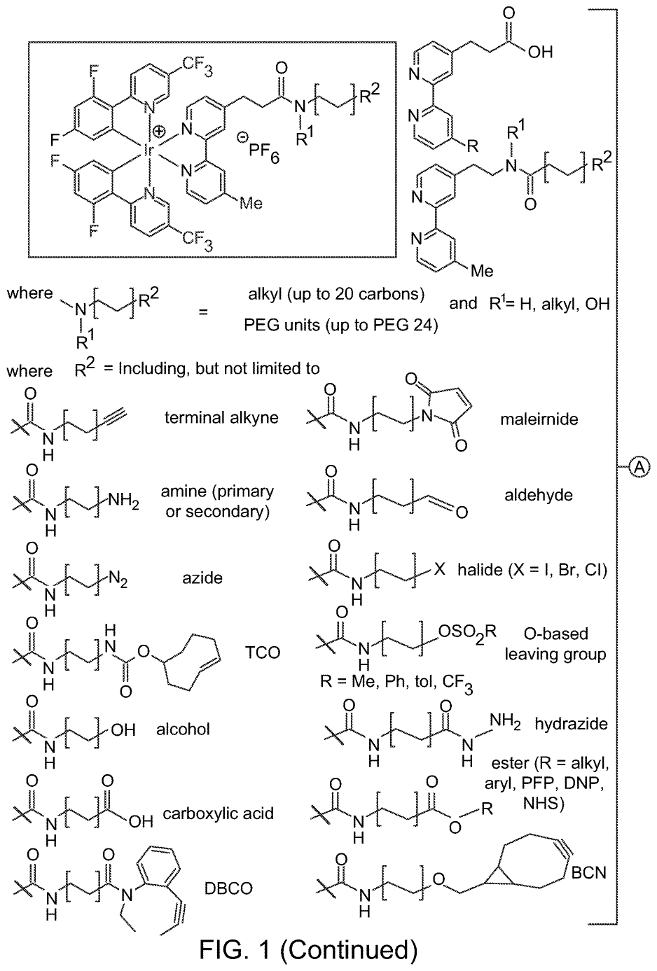

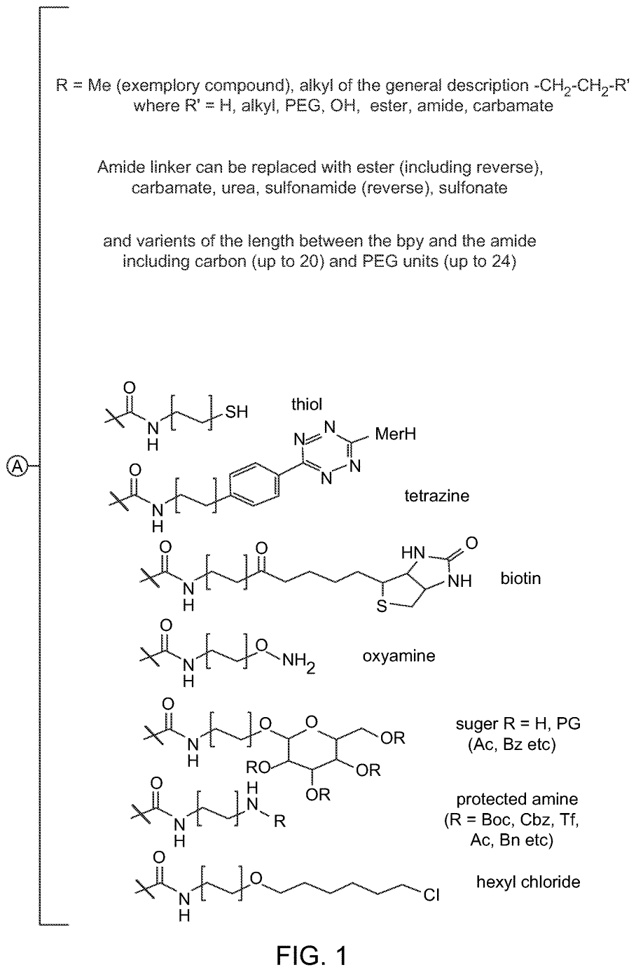

It is understood that hydrogen occupies positions on the aryl rings of Formula I in the absence of optional substituents R³-R⁷. Additionally, in some embodiments, counterion (X) can be selected from tetraalkylborate, tetrafluoroborate, tetraphenylborate, PF₆—, and chloride. Polarity of the transition metal complexes can be tailored to specific cellular environments via selection of R³-R⁷. In some embodiments, for example, one or more of R³-R⁷ are selected to exhibit hydrophobic, lipophilic, or non-polar character. In some embodiments, for example, one or more of R³-R⁷ can be alkyl, fluoro, or fluoroalkyl. Transition metal complexes described herein exhibiting hydrophobic, lipophilic, or non-polar character can be suitable for placement or passage into intracellular environments. The transition metal complexes can pass through the cellular membrane for mapping proteins in the intracellular environments according to the principles described herein. Accordingly, such transition metal complexes are cell permeable. FIG. 1 illustrates various transition metal complexes described herein. In some embodiments, L is an amide in conjunction with a polyethylene glycol (PEG) moiety for connection to R². As provided in FIG. 1, the PEG moiety can be replaced with an alkylene moiety. In some embodiments, for example, R² comprises a haloalkane or a click chemistry moiety including, but not limited to, BCN, DBCO, TCO, tetrazine, alkyne, and azide. As illustrated in FIG. 1, these click chemistries of R² can be directly coupled to the linker (L) or coupled via a heteroatom, aryl, or carbonyl.

The transition metal complex can have electronic structure for energy transfer to a protein labeling agent to produce a reactive intermediate. In some embodiments, the energy transfer is Dexter energy transfer or electron transfer. Energy transfer to the protein labeling agent can originate from an excited state of the transition metal complex electronic structure, in some embodiments. The excited state of the complex, for example, can be a singlet excited state or triplet excited state. A transition metal complex of Formula I, in some embodiments, can exhibit a long-lived triplet excited state (T₁) facilitating energy transfer to the protein labeling agent. The T₁ state can have t_{1/2} of 0.2-2 μs, for example. Transition metal complexes described herein can be photocatalytic and, in some embodiments, absorb light in the visible region or infrared region of the electromagnetic spectrum. Absorption of electromagnetic radiation can excite the transition metal complex to the Si state followed by quantitative intersystem crossing to the T₁ state. The transition metal complex can subsequently undergo short-range Dexter energy transfer to a protein labeling agent, and returned to the ground state, S₀. The energy transfer to the labeling agent activates the labeling agent for reaction with a protein or other biomolecule in the local environment of the protein substrate. The T₁ state of the transition metal complex can be greater than 60 kcal/mol, in some embodiments. The metal center, for example, can be selected from transition metals of the platinum group. The metal center can be iridium, in some embodiments.

In other embodiments, the complex may be placed in an excited state by interaction with one or more chemical species in the surrounding environment. Alternatively, energy transfer to the protein labeling agent, including electron transfer, may originate from a ground state of the complex electronic structure. Energy transfer the protein labeling agent to produce the reactive intermediate permits profiling of microenvironments local to protein substrate of ternary complexes, as described herein.

### II. Methods and Systems for Profiling Protein Degradation or Proteolysis

In another aspect, methods of characterizing protein degradation or proteolysis are described herein. In some embodiments, a method of characterizing protein degradation comprises providing a protein substrate having a transition metal complex associated therewith, and activating a protein labeling agent to a reactive intermediate with the transition metal complex. The reactive intermediate couples to an ubiquitin E3 ligase forming a complex with the protein substrate. The transition metal complex can be associated with the protein substrate by any architecture described herein, including the mechanisms described in Section I above. Moreover, the transition metal complex can be of Formula I herein and have any properties described in Section I above.

As described herein, the transition metal complex can have electronic structure for energy transfer to a protein labeling agent to produce a reactive intermediate. In some embodiments, the energy transfer is Dexter energy transfer or electron transfer. Energy transfer to the protein labeling agent can originate from an excited state of the transition metal complex electronic structure, in some embodiments. The excited state of the complex, for example, can be a singlet excited state or triplet excited state. The excited state of the transition metal complex can be generated by one or more mechanisms, including energy absorption by the complex. In some embodiments, the transition metal complex is a photocatalyst, wherein the excited state is induced by absorption of one or more photons. In other embodiments, the transition metal complex may be placed in an excited state by interaction with one or more chemical species in the surrounding environment. Alternatively, energy transfer to the protein labeling agent, including electron transfer, may originate from a ground state of the transition metal complex electronic structure. The transition metal complex can be of Formula I provided above.

The diffusion radius of the reactive intermediate can be tailored to specific microenvironment mapping (proximity-based labeling) considerations, and can be limited to the nanometer scale. In some embodiments, for example, the diffusion radius of the reactive intermediate can be less than 100 nm, less than 50 nm, less than 10 nm, less than 5 nm, less than 4 nm, less than 3 nm, or less than 2 nm prior to quenching in the surrounding environment. The diffusion radius can be 0.5 nm to 10 nm, in some embodiments. Accordingly, the reactive intermediate will react or crosslink with a protein or other biomolecule within the diffusion radius or be quenched by the surrounding environment if no protein or biomolecule is present. In this way, high resolution of the environment local to the protein substrate can be mapped via concerted effort between the transition metal complex and protein labeling agent. Additionally, the reactive intermediate can exhibit a t_{1/2} less than 5 ns, less than 4 ns, or less than 2 ns prior to quenching, in some embodiments. The reactive intermediate, for example, can exhibit a t_{1/2} less of 1-5 ns. In additional embodiments, the diffusion radius can be extended to between 5-500 nm though extension of the reactive intermediate half-life. For example, in some embodiments, the reactive intermediate can have a half-life of 1-100 μs, or greater. In some embodiments, more than one species of type of protein labeling agent can be employed in systems described herein. For example, two or more species having different diffusion radii can be employed in a system. In such embodiments, the differing diffusion radii can permit labeling at differing distances from the transition metal complex, thereby further mapping the interactome of the protein substrate of the ternary complex.

In some embodiments, the protein labeling agent can be a diazirine. Triplet energy transfer from the excited state photocatalyst can promote the diazirine to its triplet (T₁) state. The diazirine triplet under-goes elimination of N₂ to release a free triplet carbene, which undergoes picosecond-timescale spin equilibration to its reactive singlet state (t_{1/2}<1 ns) which either crosslinks with a nearby protein or is quenched in the aqueous environment. In some embodiments, the extinction coefficient of the transition metal complex is 3 to 5 orders of magnitude greater than that of the diazirine.

Any diazirine consistent with the technical principles discussed herein. Diazirine sensitization, for example, can be extended to a variety of p- and m-substituted aryltrifluoromethyl diazirines bearing valuable payloads for microscopy and proteomics applications, including free carboxylic acid, phenol, amine, alkyne, carbohydrate, and biotin groups. The diazirine can be functionalized with a marker, such as biotin. In some embodiments, the marker is desthiobiotin. The marker can assist in identification of proteins labeled by the protein labeling agent. The marker, for example, can be useful in assay results via western blot and/or other analytical techniques. Markers can include alkyne, azide, FLAG tag, fluorophore, and chloroalkane functionalities, in addition to biotin and desthiobiotin.

In additional embodiments wherein the transition metal complex is a photocatalyst, the protein labeling agent can be an azide. Triplet energy transfer from the excited state photocatalyst can promote nitrene formation from the azide. The reactive nitrene either crosslinks with a nearby protein or is quenched in the aqueous environment. Any azide operable to undergo energy transfer with eth transition metal photocatalyst for nitrene formation can be employed. In some embodiments, an azide is an aryl azide.

High resolution of the environment local to the protein substrate can be mapped via concerted effort between the transition metal complex and protein labeling agent. In this way, the ubiquitin E3 ligase forming a complex with the protein substrate can be determined. Other molecular species forming the complex or in the diffusion radius of the reactive intermediate can also be elucidated with the labeling agent, including small molecules recruiting or linking the ubiquitin E3 ligase to the protein substrate.

In some embodiments, the method further comprises coupling a small molecule to the protein substrate or coupling a small molecule to the E3 ligase, the small molecule recruiting attachment of the ubiquitin E3 ligase to provide the protein complex. The small molecule, in some embodiments, can be a synthetic molecule, a protein or peptide. The reactive intermediate, in some embodiments, may also couple to the small molecule. Structural information derived from the ubiquitin E3 ligase forming a complex with the protein substrate and/or small molecule can be employed to develop one or more additional small molecules operable to recruit/attach the ubiquitin E3 ligase to different protein substrates of interest. A library of molecular glues can be constructed for various E3 ligases and/or other molecules. The protein substrate having a transition metal complex coupled thereto in conjunction with a protein labeling agent provides a system for interrogating the local interactome of the protein substrate, including interactions with E3 ligases. In some embodiments, the protein substrate is a receptor.

Additionally, in some embodiments, methods of characterizing protein degradation or proteolysis begins with an ubiquitin E3 ligase having a transition metal complex associated therewith, and activating a protein labeling agent to a reactive intermediate with the transition metal complex. The reactive intermediate is coupled to a protein substrate forming a complex with the E3 ligase. The transition metal complex can be associated with the E3 ligase by any architecture described herein, including the mechanisms described in Section I above. Having the transition metal complex coupled to the ubiquitin E3 ligase can permit profiling of microenvironments local to the E3 ligase. In this way proteins interacting with the E3 ligase for ubiquitination can be identified. Such identification can discover previously unknown interactions, thereby expanding the use of E3 ligases for broader protein degradation applications.

The E3 ligase having a transition metal complex described herein coupled thereto in conjunction with a protein labeling agent provides a system for interrogating the local interactome of the E3 ligase, including interactions with various proteins.

### III. Methods of Mapping Intracellular Protein Interactomes

In another aspect, methods of profiling microenvironments local to an intracellular protein are described herein. In some embodiments, a method comprises forming a protein complex comprising a transition metal complex coupled to the intracellular protein, and activating a protein labeling agent to a reactive intermediate with the transition metal complex. The reactive intermediate couples to a protein or other biomolecule within a predetermined radius of the protein complex. The protein complex, transition metal complex, and protein labeling agent can have any composition and/or properties described in Sections I and II above. For example, the transition metal complex can be bound to the intracellular protein by any mechanism or architecture described in Section I above. In some embodiments, the reactive intermediate coupled to one or more proteins participating in one or more degradative pathways of the intracellular protein.

In some embodiments, the intracellular protein is a receptor. In such embodiments, the protein labeling agent is activated after molecular binding to the receptor. Molecular binding to the receptor can be binding of a receptor activator. Alternatively, molecular binding can be binding of an inhibitor or degrader of the receptor. The protein labeling agent may also be activated in the absence of any binding to the receptor.

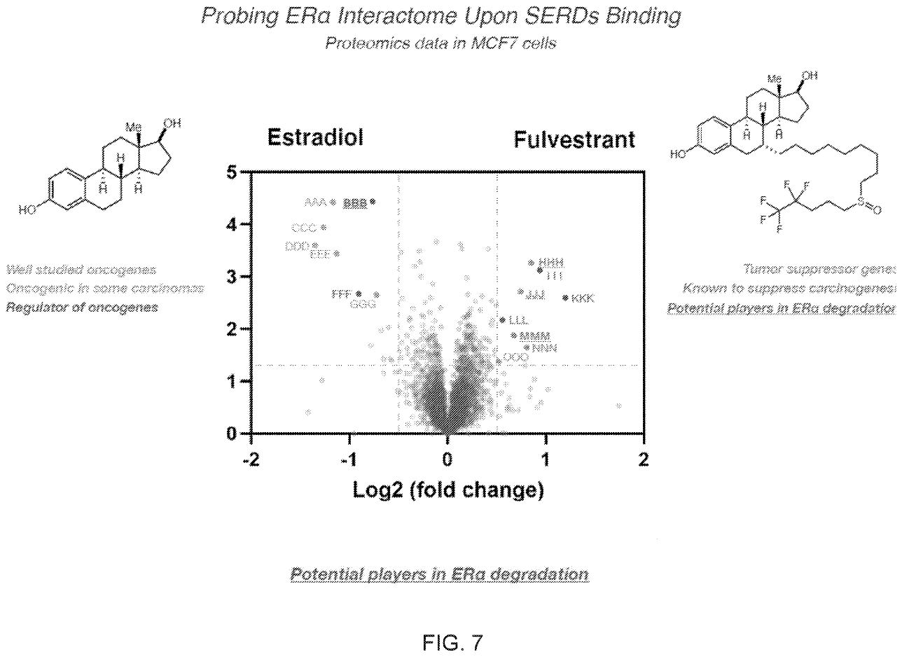

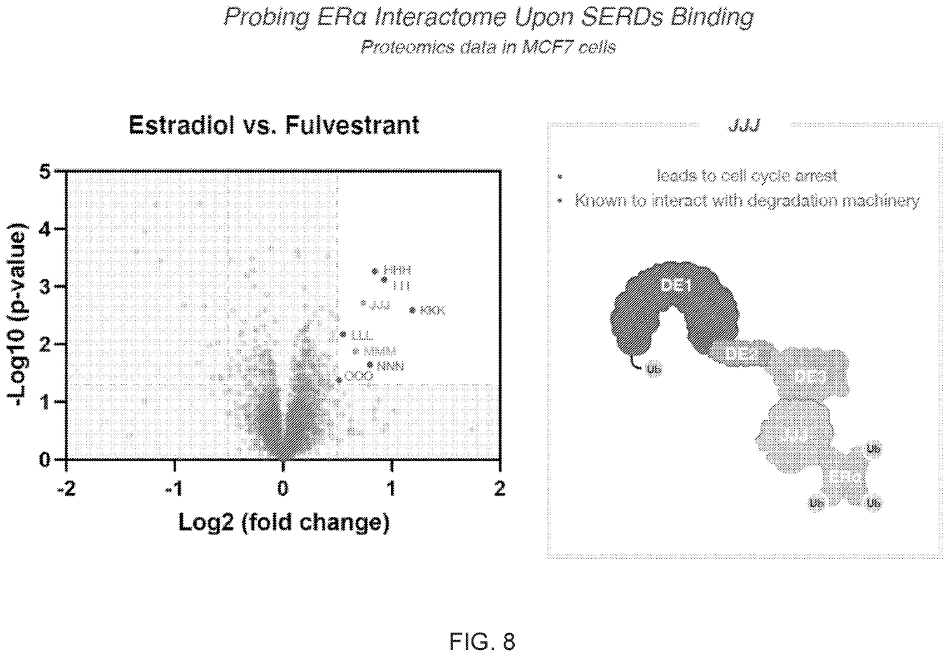

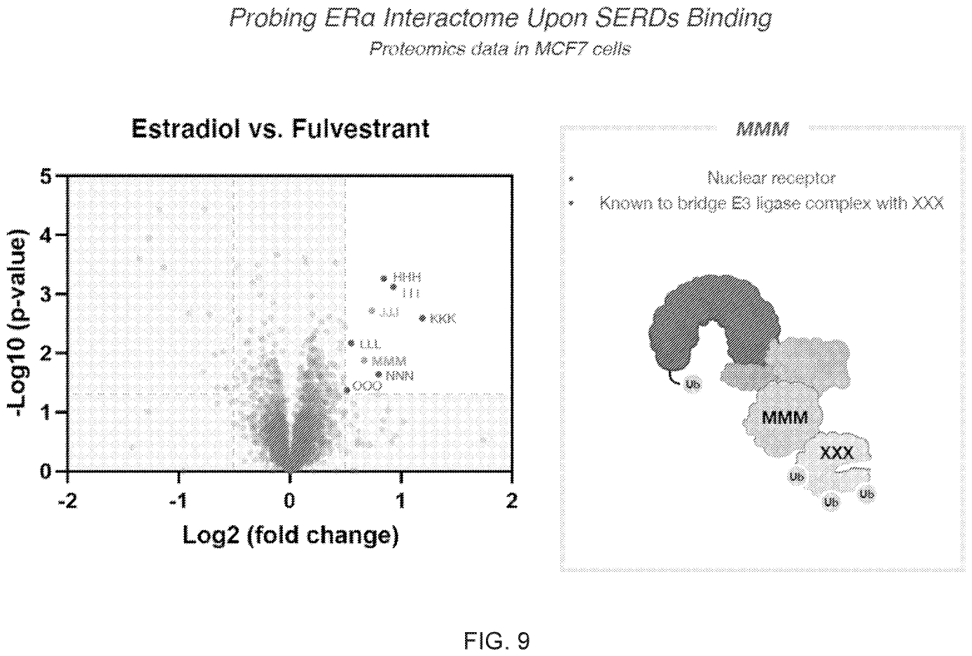

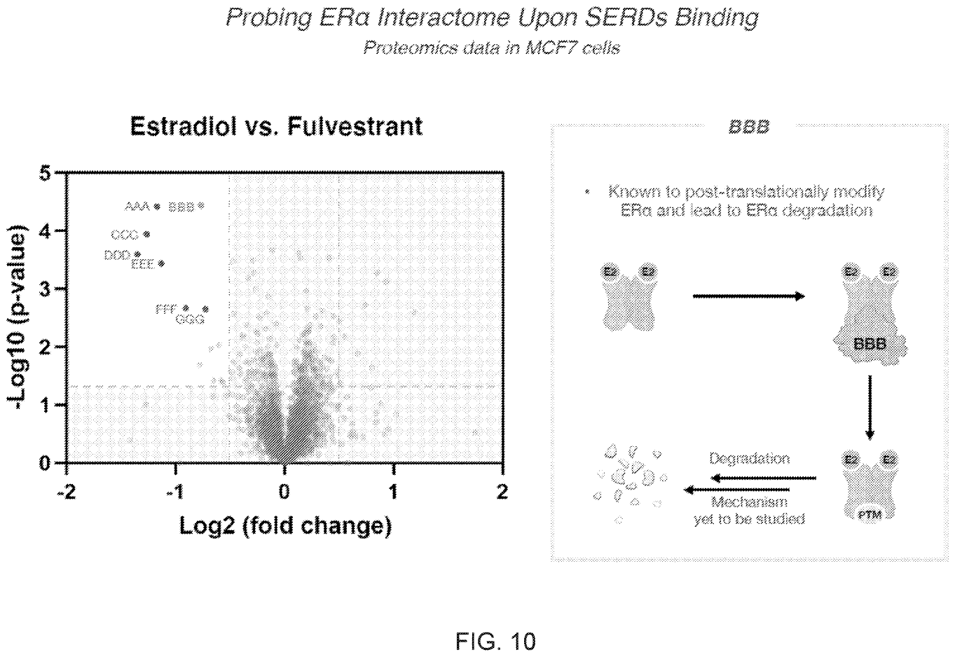

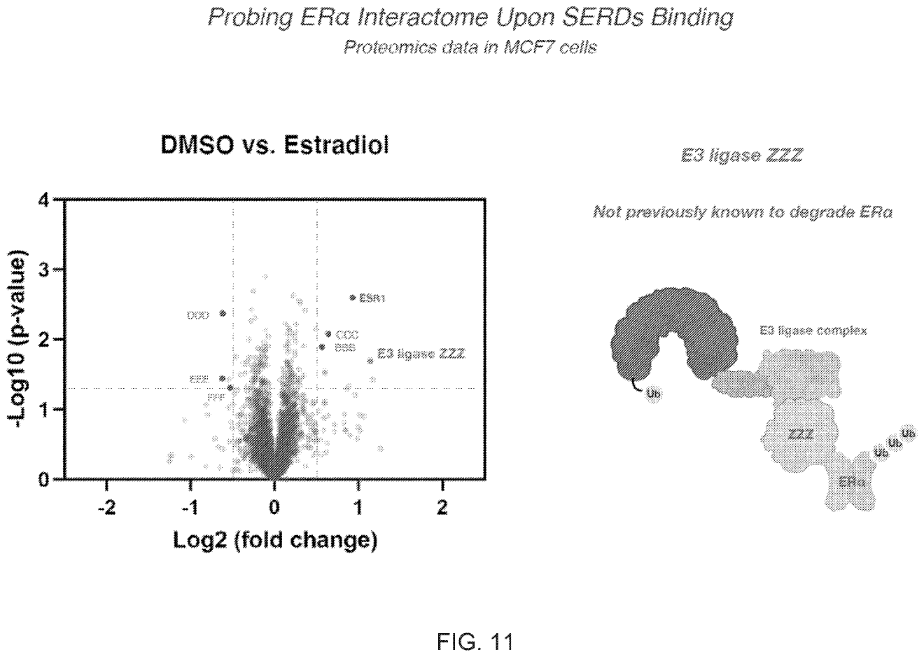

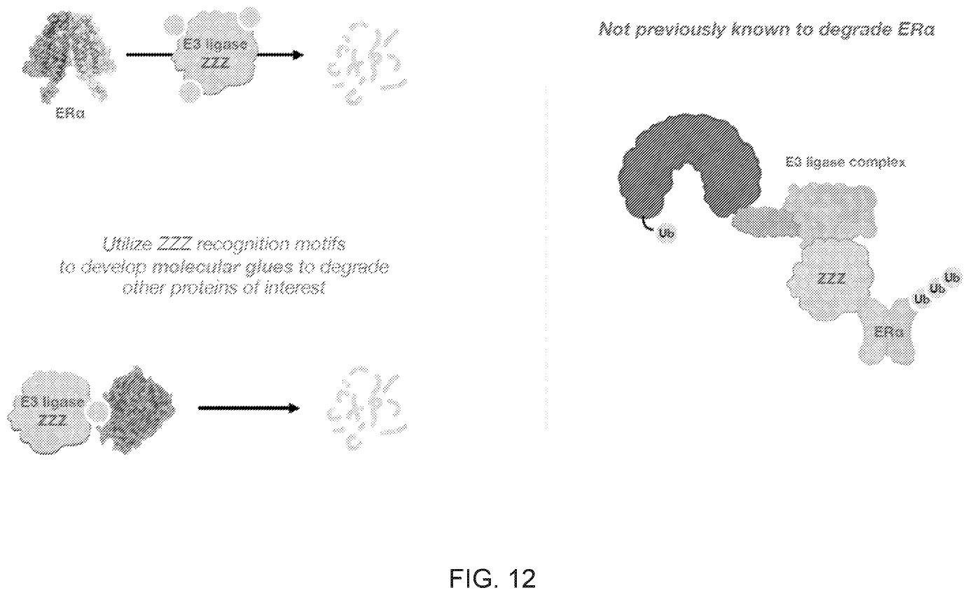

The foregoing principles are illustrated schematically in FIGS. 7-12. FIG. 7 illustrates mapping the interactome of estrogen receptor a (ERα) after separate binding of estradiol and the selective estrogen receptor degrader, fulvestrant. As provided generically in FIG. 7, the ERα interactome mapping, upon binding of estradiol, can reveal the presence of well-studied oncogenes, oncogenic proteins, and/or regulators of oncogenes. In contrast, the binding of fulvestrant can reveal the presence of tumor suppressor genes, proteins known to suppress carcinogenesis, and/or proteins involved in ERα degradative pathways. The interactome mapping upon binding of fulvestrant, for example, can identify protein species JJJ and MMM known to interact with ERα degradation machinery including, relevant E3 ligase complexes for ERα ubiquitination, as illustrated in FIG. 8 and FIG. 9, respectively. The binding of estradiol may also reveal species interacting or playing a role in the ERα degradation machinery, as illustrated in FIGS. 10 and 11. In FIG. 10, protein species BBB may be identified, which is known to post-translationally modify ERα and lead to ERα degradation. Species BBB, for example, may be a kinase participating in phosphorylated mediated degradation of ERα. Moreover, E3 ligases previously unknown to degrade ERα may be elucidated as shown in FIG. 11. This interactome mapping can be employed to develop targeted E3 ligase activators for the treatment of breast cancer and for the development of other molecular glues to degrade proteins of interest, as illustrated in FIG. 12. Example 2 herein further illustrates these principles.

These and other embodiments are further illustrated in the following non-limiting examples.

### EXAMPLE 1—TRANSITION METAL COMPLEX COUPLING TO PROTEIN

A protein-transition metal complex composite of FIG. 2 was prepared as follows. Cells (HEK243T stably expressing HaloTag-G3BP1) were seeded and grown to 95% confluency prior to labeling. The cells were incubated in DMEM media containing 5 μM of the with iridium photocatalyst of FIG. 2 for one hour. During this period, the iridium photocatalyst passes through the cell membranes and conjugates with the HaloTag-G3BP1 protein. The cells were then incubated in fresh DMEM media for one hour to remove or minimize noise from any unbound iridium catalyst.

### EXAMPLE 2—ERα INTERACTOME MAPPING IN VIEW OF ESTRADIOL/FULVESTRANT BINDING

The local interactome of ERα subsequent to binding of estradiol and fulvestrant was investigated according to compositions and methods described herein.

Each 10 cm plate of MCF7 cells stably expressing FLAG-CfaC-HA-ESR1 at 80% confluency was changed to phenol red free DMEM supplemented with 10% v/v charcoal-stripped FBS, 1% penicillin/streptomycin, and 1% L-Glutamine. After 3 days of culture, cells were treated with 10 μM TAK243 for 1 h, then co-treated with 100 nM estradiol or 100 nM fulvestrant for 4 h before harvest. The resulting cell pellets were washed 2 times with DPBS followed by hypotonic lysis using 0.6 mL of RSB buffer (10 mM Tris buffer, 15 mM NaCl, 1.5 mM MgCl₂, Roche complete EDTA-free protease inhibitors, pH 7.6, supplemented with 100 nM estradiol or 100 nM fulvestrant) for 10 min on ice. After isolating the crude nuclei using centrifugation at 400 g for 5 min at 4° C., the nuclei were resuspended in 1 mL of RSE buffer and homogenized using 10 strokes of a loose pestle Dounce homogenizer.

The nuclei were again pelleted at 400 g for 5 min at 4° C. and resuspended in 0.6 mL of cross-linking buffer (20 mM HEPES, 1.5 mM MgCl₂, 150 mM KCl, Roche complete EDTA-free protease inhibitors, pH 7.6, supplemented with 100 nM estradiol or 100 nM fulvestrant), then centrifuged again at 400 g for 5 min at 4° C. At this point, two 10 cm plate worth of pellets of the same treatment types were combined and resuspended using 400 μL cross-linking buffer with 0.3 μM of CfaN-Ir. Trans-splicing was allowed to happen for 1 h at 37° C. with rotation. The resulting pellets were washed 3× using 400 μL cross-linking buffer before the addition of 500 μM biotin-diazirine in 200 μL cross-linking buffer, and irradiation was performed for 3 min at 4° C. using 100% intensity of blue light with Penn PhD Photoreactor M2. The irradiated nuclei were washed again 2 times with 400 μL cross-linking buffer prior to the addition of LB3 buffer (10 mM Tris, 100 mM NaCl, 1 mM EDTA, 0.5 mM EGTA, 0.1% sodium deoxycholate, 0.5% sodium lauroyl sarcosinate, Roche complete EDTA-free protease inhibitors, pH 7.5) for lysis 10 min on ice. The pellets were sonicated twice using a Branson probe tip sonicator for 10 seconds at 35% amplitude and then centrifuged at 18000 g for 15 min at 4° C. The supernatant was obtained as nuclear lysate and the protein concentration was determined using BCA assay.

All replicates were diluted to 0.5 mg/mL using binding buffer (25 mM Tris, 150 mM NaCl, 0.2% v/v NP-40, pH 7.5). Lysate was loaded onto 25 μL of prewashed magnetic Sepharose streptavidin beads for 18 h at 4° C. with end-over-end rotation. The beads were then washed with 3 times 1% w/v SDS in DPBS, twice 1M NaCl in DPBS, once 10% ethanol in DPBS, and 3 times 50 mM ammonium bicarbonate in water. The washed beads were transferred to a fresh Lo-bind tube using 500 μL of 50 mM ammonium bicarbonate, with as much supernatant removed as possible. The resulting bead pellets were resuspended in 30 μL of 50 mM ammonium bicarbonate with 0.4 ug of trypsin and incubated overnight at 37° C. with rotation. The resulting supernatant was collected using centrifugation and filtered using Spin-X Centrifuge Tube Filter with 0.22 μM cellulose acetate. The resulting eluent is ready for mass spectrometry analysis using TimsTOF.

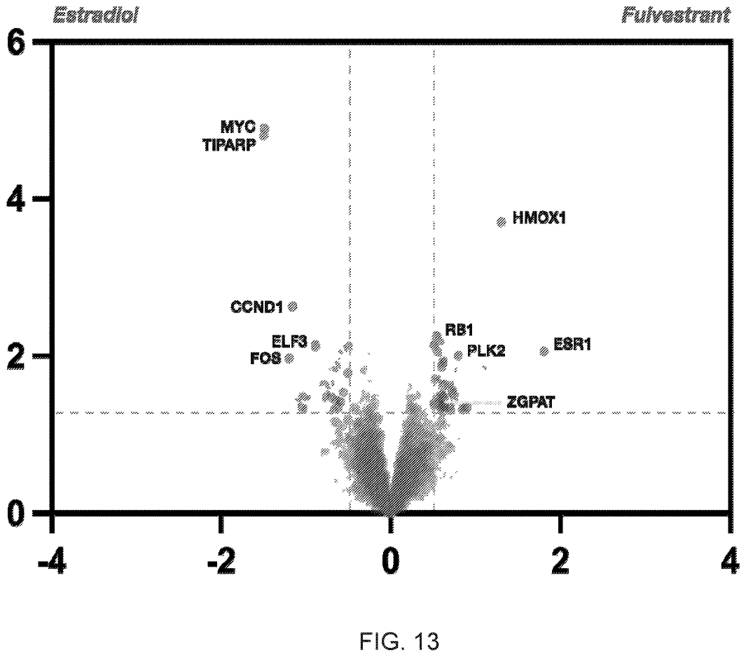

FIG. 13 is a volcano plot detailing the species identified in the interactome mapping post binding of estradiol and fulvestrant. The volcano plot is a composite of the result obtain from the independent runs of binding estradiol and fulvestrant.

Various embodiments of the invention have been described in fulfillment of the various objects of the invention. It should be recognized that these embodiments are merely illustrative of the principles of the present invention. Numerous modifications and adaptations thereof will be readily apparent to those skilled in the art without departing from the spirit and scope of the invention.

1. A protein complex comprising:
a protein substrate having an ubiquitin E3 ligase associated therewith, wherein a transition metal complex is coupled to the protein substrate.

2. The protein complex of claim 1, wherein the transition metal complex is bonded to a haloalkane dehalogenase, the haloalkane dehalogenase coupled to the protein substrate.

3. The protein complex of claim 1, wherein the transition metal complex is bonded to an unnatural amino acid of the protein substrate.

4. The protein of claim 3, wherein the unnatural amino acid comprises a click chemistry moiety.

5. The protein complex of claim 4, wherein the unnatural amino acid and transition metal complex are coupled by click chemistry.

6. The protein complex of claim 1, wherein the transition metal complex is coupled to the protein substrate via protein-trans splicing, the transition metal complex being initially coupled to a split intein.

7. The protein complex of claim 6, wherein the split intein is an N-intein.

8. The protein complex of claim 6, wherein the split intein is an C-intein.

9. The protein complex of claim 1 further comprising one or more small molecules between the protein substrate and E3 ligase.

10. The protein complex of claim 1, wherein the transition metal complex is of Formula (I): 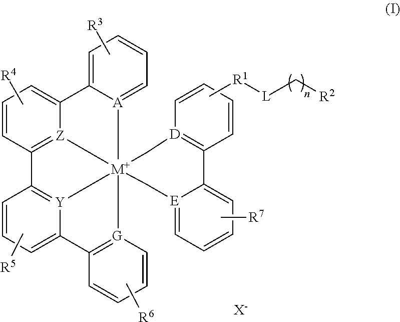
wherein M is a transition metal;
wherein A, D, E, G, Y and Z are independently selected from C and N;
wherein R³-R⁷ each represent one to four optional ring substituents, each of the one to four optional ring substituents independently selected from the group consisting of alkyl, heteroalkyl, haloalkyl, haloalkenyl, halo, hydroxy, alkoxy, amine, amide, ether, —C(O)O⁻, —C(O)OR⁸, and —R⁹OH, wherein R⁸ is selected from the group consisting of hydrogen and alkyl, and R⁹ is alkyl;
wherein R¹ is selected from the group consisting of a direct bond, alkylene, alkenylene, cycloaklylene, cycloalkenylene, arylene, heteroalkylene, heteroalkenylene, heterocyclene, and heteroarylene;
wherein L is an optional linking moiety selected from the group consisting of amide, ester, sulfonamide, sulfonate, carbamate, and urea; and
R² is selected from the group consisting of alkyne, amine, protected amine, azide, hydrazide, aryl, heteroaryl, cycloalkyl, cycloalkenyl, cycloalkylnyl, heterocyclyl, hydroxy, carboxyl, halo, alkoxy, maleimide, —C(O)H, —C(O)OR⁸, —OS(O₂)R⁹, thiol, biotin, oxyamine, and haloalkyl, wherein R⁸ and R⁹ are independently selected from the group consisting of alkyl, haloalkyl, aryl, haloaryl, N-succinimidyl, and N-succinimidyl ester; and wherein X⁻ is a counterion, and n is an integer from 0 to 20.

11. A method of characterizing protein degradation comprising:
providing a protein substrate having a transition metal complex associated therewith;
activating a protein labeling agent to a reactive intermediate with the transition metal complex; and
coupling the reactive intermediate to an ubiquitin E3 ligase forming a complex with the protein substrate.

12. The method of claim 11 further comprising coupling a small molecule to the protein substrate, the small molecule recruiting attachment of the ubiquitin E3 ligase to provide a protein complex.

13. The method of claim 12 further comprising developing one or more additional small molecules operable to recruit the ubiquitin E3 ligase to different protein substrates.

14. The method of claim 11, wherein the transition metal complex is of the formula: 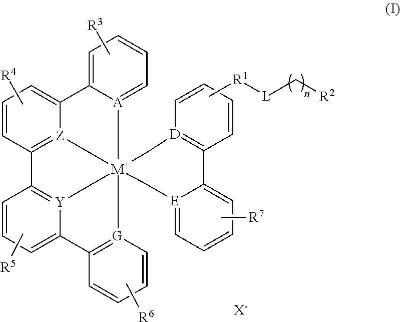
wherein M is a transition metal;
wherein A, D, E, G, Y and Z are independently selected from C and N;
wherein R³-R⁷ each represent one to four optional ring substituents, each of the one to four optional ring substituents independently selected from the group consisting of alkyl, heteroalkyl, haloalkyl, haloalkenyl, halo, hydroxy, alkoxy, amine, amide, ether, —C(O)O⁻, —C(O)OR⁸, and —R⁹OH, wherein R⁸ is selected from the group consisting of hydrogen and alkyl, and R⁹ is alkyl;
wherein R¹ is selected from the group consisting of a direct bond, alkylene, alkenylene, cycloaklylene, cycloalkenylene, arylene, heteroalkylene, heteroalkenylene, heterocyclene, and heteroarylene;
wherein L is an optional linking moiety selected from the group consisting of amide, ester, sulfonamide, sulfonate, carbamate, and urea; and
R² is selected from the group consisting of alkyne, amine, protected amine, azide, hydrazide, aryl, heteroaryl, cycloalkyl, cycloalkenyl, cycloalkylnyl, heterocyclyl, hydroxy, carboxyl, halo, alkoxy, maleimide, —C(O)H, —C(O)OR⁸, —OS(O₂)R⁹, thiol, biotin, oxyamine, and haloalkyl, wherein R⁸ and R⁹ are independently selected from the group consisting of alkyl, haloalkyl, aryl, haloaryl, N-succinimidyl, and N-succinimidyl ester; and wherein X is a counterion, and n is an integer from 0 to 20.

15. The method of claim 12, wherein the small molecule is also labeled by the protein labeling agent via the reactive intermediate.

16. The method of claim 11, wherein the transition metal complex is bonded to a haloalkane dehalogenase, the haloalkane dehalogenase coupled to the protein substrate.

17. The method of claim 11, wherein the transition metal complex is bonded to an unnatural amino acid of the protein substrate.

18. The method of claim 17, wherein the unnatural amino acid comprises a click chemistry moiety.

19. The method of claim 11, wherein the transition metal complex is coupled to the protein substrate via protein-trans splicing, the transition metal complex being initially coupled to a split intein.

20. The method of claim 11, wherein the protein substrate is a receptor.

21. A method of profiling microenvironments local to an intracellular protein comprising:
forming a protein complex comprising a transition metal complex coupled to the intracellular protein;
activating a protein labeling agent to a reactive intermediate with the transition metal complex; and
coupling the reactive intermediate to a protein or other biomolecule within a predetermined radius of the protein complex.

22. The method of claim 21, wherein forming the protein complex comprises coupling the transition metal complex to a haloalkane dehalogenase, the haloalkane dehalogenase coupled to the intracellular protein.

23. The method of claim 21, wherein forming the protein complex comprises coupling the transition metal complex to an unnatural amino acid of the intracellular protein.

24. The method of claim 23, wherein the transition metal complex is coupled to the unnatural amino acid via click chemistry.

25. The method of claim 21, wherein the transition metal complex is coupled to the intracellular protein via protein-trans splicing, the transition metal complex being initially coupled to a split intein.

26. The method of claim 25, wherein the split intein is an N-intein.

27. The method of claim 25, wherein the split intein is a C-intein.

28. The method of claim 21, the method of claim 21, wherein the reactive intermediate couples to a protein participating in a degradative pathway of the intracellular protein.

29. The method of claim 28, wherein the reactive intermediate couples to an E3 ligase of the intracellular protein.

30. The method of claim 21, wherein the intracellular protein is a receptor.

31. The method of claim 30, wherein the protein labeling agent is activated after molecular binding to the receptor.

32. The method of claim 31, wherein the molecular binding comprises binding an activator of the receptor.

33. The method of claim 31, wherein the molecular binding comprises binding an inhibitor or degrader of the receptor.
